(19) United States
(12) Patent Application Publication
(10) Pub. No.: US 2024/0104739 A1
(43) Pub. Date: Mar. 28, 2024

(54) COMPUTER VISION FOR ANALYZING HANDWRITING KINEMATICS

(71) Applicant: RON NACHUM, VIENNA, VA (US)

(72) Inventor: RON NACHUM, VIENNA, VA (US)

(21) Appl. No.: 17/953,001

(22) Filed: Sep. 26, 2022

(51) Int. Cl. G06T 7/00; A61B 5/00; G06T 7/73; G06V 10/25; G06V 10/44; G06V 10/774; G06V 20/40; G06V 40/20

(52) U.S. Cl. CPC G06T 7/0016; A61B 5/4082; A61B 5/4088; G06T 7/74; G06V 10/25; G06V 10/44; G06V 10/774; G06V 20/41; G06V 20/46; G06V 40/28; G06T 2207/10016; G06T 2207/10024; G06T 2207/20081; G06T 2207/20084; G06T 2207/30004; G06T 2207/30196

(57) ABSTRACT

A system for analyzing handwriting kinematics includes a memory that stores executable instructions and a processor that executes the instructions. When executed by the processor, the instructions cause the system to implement a process that includes: receiving RGB video data of a subject performing handwriting; extracting features from the RGB video data, and analyzing the RGB video data for handwriting characteristics based on the features extracted from the RGB video data.

### BACKGROUND

The current diagnostic process for neurodegenerative diseases (NDs), such as Alzheimer's Disease (AD) and Parkinson's Disease (PD), is complex and taxing on patients. The current diagnostic process involves multiple specialists relying on their judgment and leveraging a variety of approaches such as mental status exams, cognitive assessment, and brain imaging to build a case and rule out alternative causes for symptoms. The current diagnostic process is often delayed two to three years after symptom onset and takes several months to reach a conclusion. Because of these barriers to diagnosis, up to 50% of patients with neurodegenerative diseases are not diagnosed during their lifetime. Even for patients who receive a diagnosis, an accurate conclusion is not guaranteed; studies have shown that the current clinical diagnostic process for neurodegenerative diseases is typically only 75-80% accurate.

Fine motor movement is a demonstrated biomarker for many health conditions that are especially difficult to diagnose early and require sensitivity to change in order to monitor over time. This is especially true for neurodegenerative diseases, including Alzheimer's Disease and Parkinson's Disease, both of which are associated with early changes in handwriting and fine motor skills. Kinematic analysis of handwriting is an emerging method for assessing fine motor movement ability, with data typically collected by digitizing tablets. However, digitizing tables are often expensive, unfamiliar to patients, and provide limited scope of collectible data.

Digitizing tablets are capable of collecting both pen position and pressure data. Currently, computer vision-based systems are unable to collect high accuracy pressure data, which has been shown to increase classification accuracy of neurodegenerative disease by 5-10% when combined with kinematic features. However, digitizing tablets are limited in their scope of data collection compared to computer vision with computer vision-based systems providing more types of data collection.

### SUMMARY

According to an aspect of the present disclosure, a system for analyzing handwriting kinematics includes a memory that stores executable instructions and a processor that executes the instructions. When executed by the processor, the instructions cause the system to implement a process that includes: receiving RGB video data of a subject performing handwriting; extracting features from the RGB video data, and analyzing the RGB video data for handwriting characteristics based on the features extracted from the RGB video data.

According to another aspect of the present disclosure, a method for analyzing handwriting kinematics includes receiving RGB video data of a subject performing handwriting; extracting features from the RGB video data, and analyzing the RGB video data for handwriting characteristics, using a machine learning model, based on the features extracted from the RGB video data.

According to another aspect of the present disclosure, a computer-readable medium stores instructions which, when executed by a processor, implement a process. The process includes receiving RGB video data of a subject performing handwriting; extracting features from the RGB video data, and analyzing the RGB video data for handwriting characteristics based on the features extracted from the RGB video data.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1A illustrates an electronic device for analyzing handwriting kinematics, in accordance with a representative embodiment.

FIG. 1B illustrates a system for analyzing handwriting kinematics, in accordance with a representative embodiment.

FIG. 1C illustrates another system for analyzing handwriting kinematics, in accordance with a representative embodiment.

FIG. 2A illustrates a method for analyzing handwriting kinematics, in accordance with a representative embodiment.

FIG. 2B illustrates another method for analyzing handwriting kinematics, in accordance with a representative embodiment.

FIG. 2C illustrates another method for analyzing handwriting kinematics, in accordance with a representative embodiment.

FIG. 3 illustrates a process for analyzing handwriting kinematics, in accordance with a representative embodiment.

FIG. 4A illustrates an overview of a machine learning system for analyzing handwriting kinematics, in accordance with a representative embodiment.

FIG. 4B illustrates a first stage of a machine learning system for analyzing handwriting kinematics consistent with the overview in FIG. 4A, in accordance with a representative embodiment.

FIG. 4C illustrates a second stage of a machine learning system for analyzing handwriting kinematics consistent with the overview in FIG. 4A, in accordance with a representative embodiment.

FIG. 4D illustrates a third stage of a machine learning system for analyzing handwriting kinematics consistent with the overview in FIG. 4A, in accordance with a representative embodiment.

FIG. 5 illustrates a relative comparison of a computer vision-based system for analyzing handwriting kinematics to a digitizing tablet control system for analyzing handwriting kinematics, in accordance with a representative embodiment.

FIG. 6 illustrates a computer system, on which a method for analyzing handwriting kinematics is implemented, in accordance with another representative embodiment.

### DETAILED DESCRIPTION

In the following detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

As described herein, a computer vision-based system for capturing and analyzing characteristics of handwriting kinematics may be provided using a commodity camera and RGB video. A result of this approach is an accurate, accessible, and informative alternative to digitizing tablets which is usable in early disease diagnosis and monitoring, as well as more broadly in all applications of capturing handwriting or fine motor movement ability. The teachings herein provide an ability to extract handwriting kinematic features through processing of RGB video data captured by commodity cameras, such as those in smartphones.

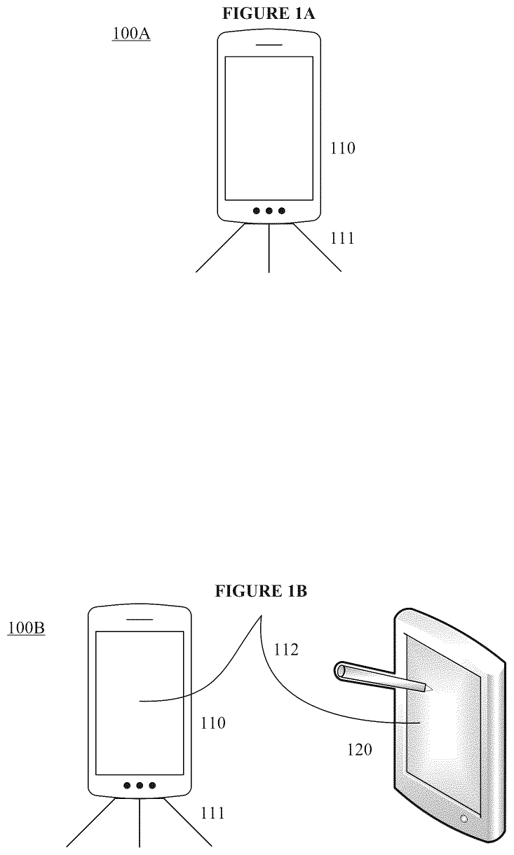

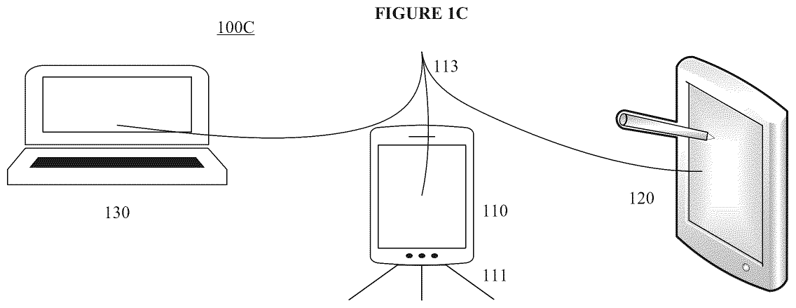

FIG. 1A illustrates an electronic device for analyzing handwriting kinematics, in accordance with a representative embodiment.

In FIG. 1A, a system 100A includes an electronic device 110 which is fixed to a stand 111. The electronic device 110 may be configured to implement the computer vision for analyzing characteristics of handwriting kinematics described herein. Computer vision-based systems are capable of quantifying hand pose and body movements and also classifying pen grip types, which have potential to further improve diagnostic assessment accuracy and require further research to support their use. For example, hand pose and pen grip type can be quantified using Google's MediaPipe library for hand landmark detection.

The electronic device 110 may be or include a commodity camera which can capture RGB video of a subject performing handwriting, and may be configured to analyze the captured handwriting. The system 100A is a computer vision-based system implemented using the electronic device 110 The system 100A enables quantification of fine motor movements and offers a fast, easy-to-use, and more widely accessible screening solution due to the pervasiveness of cameras in smartphones and laptops.

They system 100A is provided to capture handwriting kinematic information with commodity cameras. Since commodity cameras capture frames at a lower frequency (typically 30 or 60 Hz) compared to sampling rate of digitizing tablets (typically 100 Hz), the viability of lower-frequency kinematic data for diagnostic assessments using machine learning has been investigated and confirmed. The investigative process used to confirm the viability of commodity cameras for such diagnostic assessments includes down-sampling the PaHaW dataset of handwriting movements captured by a digitizing tablet and training classifiers on the resultant information to assess their accuracy. The PaHaW dataset consists of digitizing tablet data of 8 different handwriting tasks from 38 healthy controls (HCs) and 37 Parkinson's Disease patients (total 75 individuals).

Accuracy of extracted kinematic data from videos taken by the electronic device 110 and classification accuracy of resultant diagnostic assessments has also been investigated and confirmed. To best determine accuracy and statistically assess the developed computer vision-based system for kinematic data extraction, handwriting tasks may be simultaneously captured in a video format by the electronic device 110 (e.g., using a smartphone camera) and quantified by a Wacom Intous Medium digitizing tablet. These synchronized data streams enable comparison of handwriting kinematics captured by the computer vision-based system and the digitizing tablet.

By way of comparison using an electronic device 110, for example, 214 handwriting movements may be captured from a single healthy test subject to demonstrate feasibility of extracting kinematic information from videos. Measured tasks may include Archimedean spiral drawing (124 videos), tracing of 1's and e's (60 videos), and tracing of words (30 videos) on the PaHaW study writing template. The collected position data may be utilized at the original sampling frequency of 100 Hz, typical of digitizing tablets, and also at down-sampled frequencies of 30 Hz and 60 Hz, which are typical of commodity cameras. The resultant kinematic data may then be filtered with a Gaussian filter with a sigma value of, for example, 5.

The stand 111 may be a tripod. Notably, the electronic device 110 is shown in FIG. 1A without a digitizing tablet though, as explained below with respect to embodiments based on FIG. 1B and FIG. 1C, the electronic device 110 may also be used with a digitizing tablet 120 without departing from the spirit of the teachings herein.

FIG. 1B illustrates a system for analyzing handwriting kinematics, in accordance with a representative embodiment.

In FIG. 1B, a system 100B includes the electronic device 110 again fixed to the stand 111 and now connected to a digitizing tablet 120 via a wire 112. The handwriting may be performed on the digitizing tablet 120 and RGB video of the subject performing handwriting may be captured by the electronic device 110.

Digitizing tablets such as the digitizing tablet 120 are currently used to collect data for studies. The data received from the digitizing tablet 120 may include data of characteristics of the handwriting performed by a subject. Digitizing tablets are expensive and can often be inaccessible in poor-resource health systems or telemedicine settings due to their cost. Furthermore, since the use of electronic pens required for digitizing tablets may be unfamiliar to patients, completion of a time-consuming training phase may be required to acquaint patients with their use. Digitizing tablets collect strictly pen position and pressure, and are unable to capture other available data (e.g., hand pose) that could improve diagnostic accuracy. In embodiments based on FIG. 1B, data from the digitizing tablet 120 may also be received by the electronic device 110 and integrated with results of processing the RGB video to implement the teachings herein. The digitizing tablet 120 may send data of the pen position and pressure in the handwriting to the electronic device 110.

In FIG. 1A and FIG. 1B, the electronic device 110 is an element of systems for analyzing handwriting kinematics. The electronic device 110 includes at least a memory that stores executable instructions, and a processor that executes the instructions. When executed by the processor, the instructions cause the systems in FIG. 1A and FIG. 1B to implement a process that includes receiving RGB video data of a subject performing handwriting; extracting features from the RGB video data, and analyzing the RGB video data for handwriting characteristics based on the features extracted from the RGB video data.

FIG. 1C illustrates another system for analyzing handwriting kinematics, in accordance with a representative embodiment.

In FIG. 1C, a system 100C includes the electronic device 110 again fixed to the stand 111 and again connected to the digitizing tablet 120, now via a wire 113. The electronic device 110 is also now connected to a mobile computer 130. The system 100C in FIG. 1C consists of a digitizing tablet 120 overlaid with a writing template and connected to a laptop as the mobile computer 130 as well as a smartphone as the electronic device 110 on a small tripod as the stand 111. The handwriting may be performed on the digitizing tablet 120, and RGB video of the subject performing handwriting may be captured by the electronic device 110. In embodiments based on FIG. 1C, data from the digitizing tablet 120 may also be received by the mobile computer 130 and integrated with results of processing the RGB video to implement the teachings herein. The digitizing tablet 120 may send data of the pen position and pressure in the handwriting to the mobile computer 130. The electronic device 110 may send the RGB video of the subject performing handwriting to the mobile computer 130.

In FIG. 1C, the mobile computer 130 and the electronic device 110 are each elements of a system 100C for analyzing characteristics of handwriting kinematics. The mobile computer 130 includes at least a memory that stores executable instructions, and a processor that executes the instructions. When executed by the processor, the instructions cause the mobile computer 130 to implement a process that includes receiving RGB video data of a subject performing handwriting; extracting features from the RGB video data, and analyzing the RGB video data for handwriting characteristics based on the features extracted from the RGB video data.

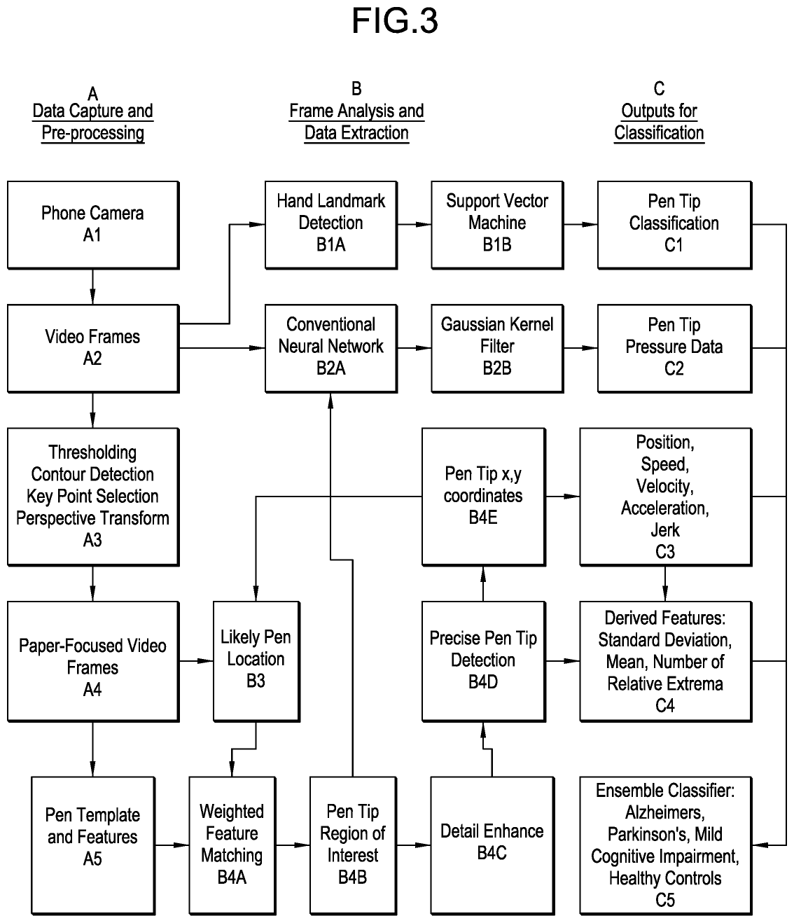

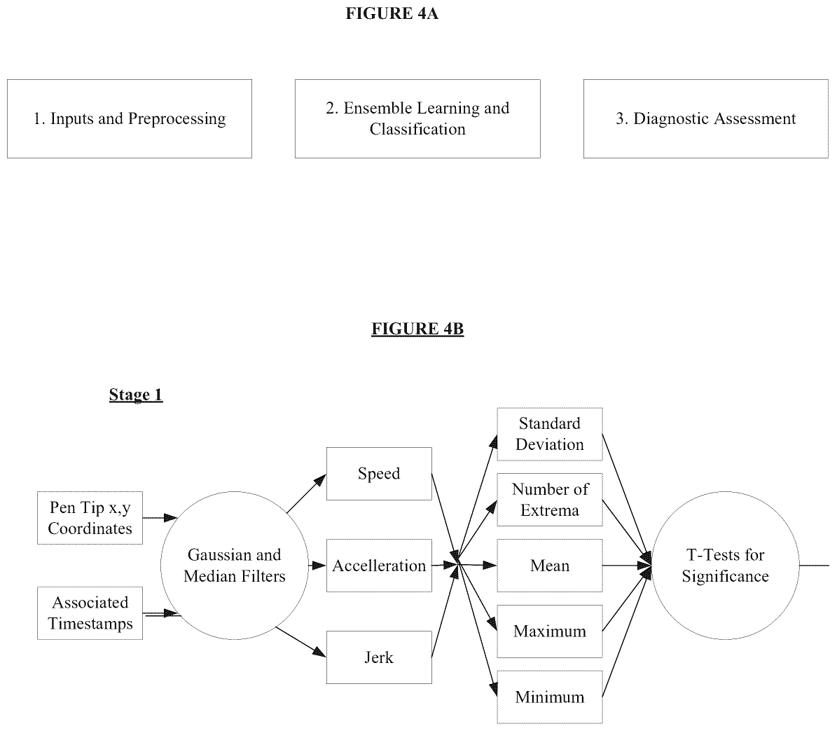

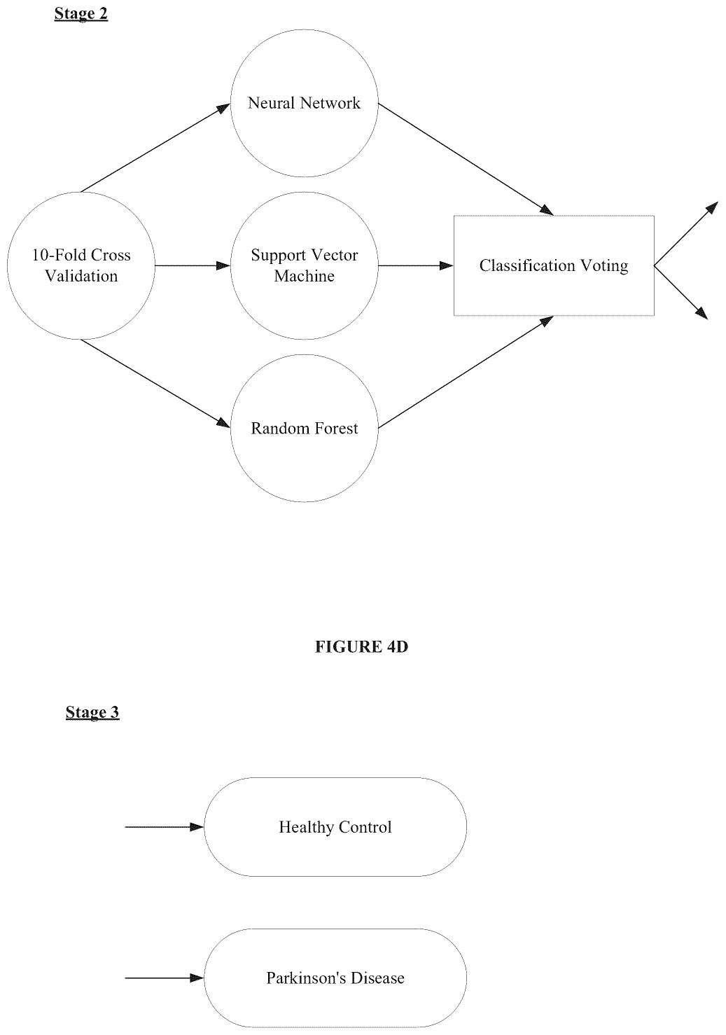

In embodiments based on or similar mostly to FIG. 1A or FIG. 1B, analysis for handwriting kinematics is performed by the electronic device 110. In embodiments based on or similar mostly to FIG. 1C, analysis for handwriting kinematics is performed by the mobile computer 130. The computer vision-based data collection systems of FIG. 1A, FIG. 1B and FIG. 1C each consist of several different structures to extract diagnostic information, largely involving use of a recurrent system for determining pen position. An example framework for the entire computer vision-based system described herein is outlined in FIG. 3. Additionally, an overview of a machine learning system for providing patient diagnostic assessment based on kinematic features of handwriting movements is illustrated in FIG. 4A and detailed in stages in FIG. 4B, FIG. 4C and FIG. 4D.

Embodiments based on FIG. 1A, FIG. 1B and FIG. 1C provide setups to collect synchronized data from smartphone videos and/or tablet videos (e.g., from the electronic device 110). The accuracy of such computer vision-based systems is readily assessed using statistical comparisons. The computer vision-based system's accuracy may be tested through direct comparison to data produced by digitizing tablets during common handwriting tasks.

Extraction and processing of handwriting kinematic data using setups based on FIG. 1A, FIG. 1B or FIG. 1C may be implemented with computer vision and machine learning implemented by the electronic device 110 and/or the mobile computer 130 or another computer connected to the electronic device 110. The collected handwriting kinematic data may, in turn, be analyzed to objectively and quantitatively differentiate between healthy individuals and patients with neurogenerative diseases, including Alzheimer's Disease and Parkinson's Disease, as well as other diseases with biomarkers displayed in fine motor movement. The computer vision-based systems and methods based on FIG. 1A, FIG. 1B and/or FIG. 1C may have many applications including providing widespread diagnostic access in low-income areas and resource-poor health systems, and may be used as an accessible form of long-term disease monitoring through telemedicine. Quantification and kinematic analysis of fine motor movements has applications for providing diagnostic assessments, as well as change over time of a previously-diagnosed disease that can be used for long-term monitoring and treatment response. Moreover, kinematic analysis of fine motor movements is applicable to assessing other health conditions with biomarkers displayed in fine motor movement, including strokes and early developmental disorders, as well as depression and anxiety.

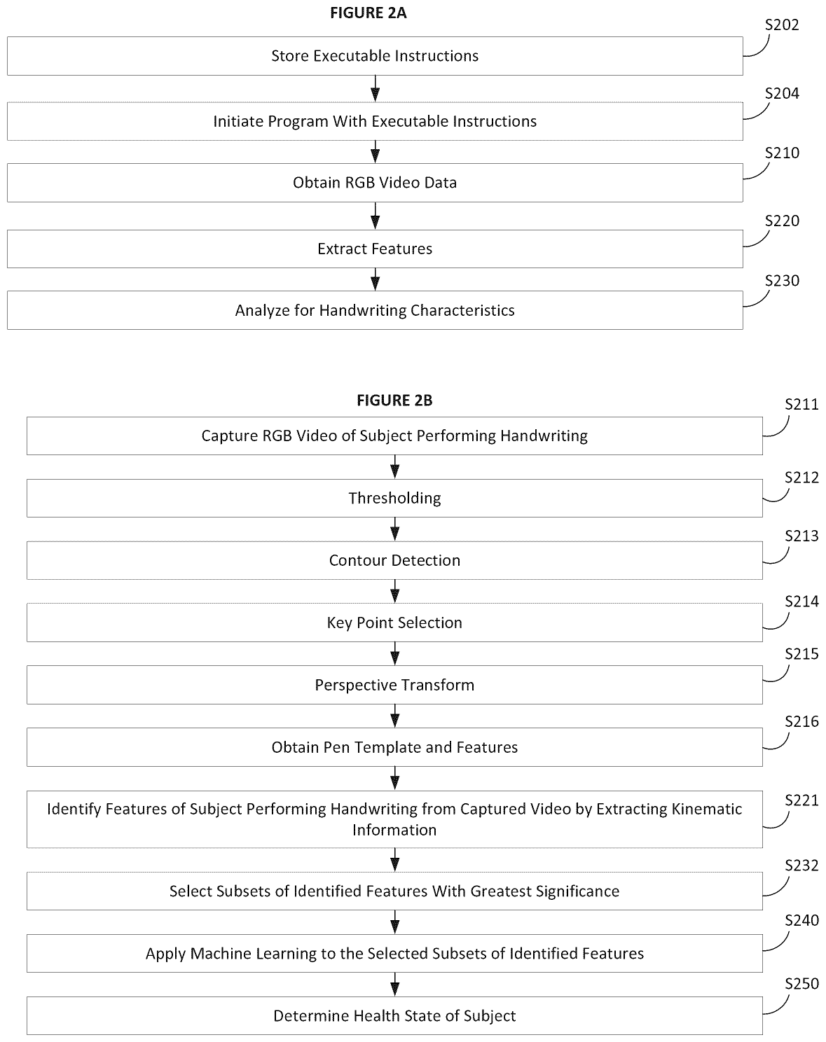

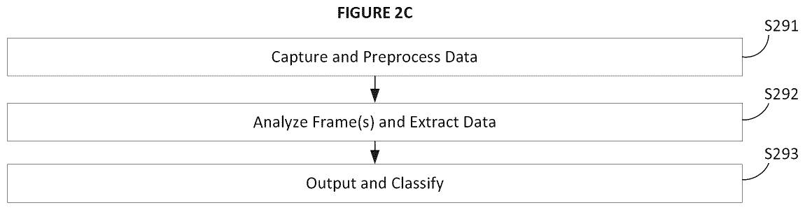

FIG. 2A illustrates a method for analyzing handwriting kinematics, in accordance with a representative embodiment.

The method of FIG. 2A may be considered an overview method for some embodiments described herein. At S202, executable instructions are stored, such as in a memory of the electronic device 110 and/or in a memory of the mobile computer 130.

At S204, a program with the executable instructions is initiated. For example, the electronic device 110 may activate a program to capture RGB video data. When the analysis for handwriting kinematics is performed by the electronic device 110, the program activated at S204 may be an analysis program, and the analysis program may be configured to take control of a commodity camera of the electronic device 110 to capture the RGB video data when appropriate. When the analysis for handwriting kinematics is performed by the mobile computer 130, separate programs may be initiated at S204 on the electronic device 110 and the mobile computer 130. The program initiated on the electronic device 110 may be configured to capture the RGB video data under the control of the electronic device 110, or under the control of the mobile computer 130 when the mobile computer 130 implements the analysis program described herein.

At S210, RGB video data is obtained. The RGB video may be obtained by the electronic device 110, and either retained for analysis by the electronic device 110 as in FIG. 1A and FIG. 1B or transmitted to the mobile computer 130 and received by the mobile computer 130 as in FIG. 1C.

At S220, features from the RGB video data are extracted. An important objective of the computer vision-based system for quantifying fine motor movements, in addition to producing vision-specific features, is to extract kinematic information with accuracy comparable to that collected by digitizing tablets. Extracting such kinematic information requires pen tip x and y coordinates tagged with timestamps. The features from the RGB video data may be or include handwriting kinematic features. Handwriting tasks captured in the RGB video data may be used to assess fine motor movement ability. Specific handwriting tasks include tracing of Archimedean spirals and cursive ‘1’s and ‘e’s, as well as writing of words and short sentences. Movement of the pen's position is tracked during performance of the specific handwriting tasks, and the tracking of movement of the pen's position may be used to compute speed, acceleration, and jerk.

At S240, the RGB video data is analyzed for handwriting characteristics based on the features extracted from the RGB video data. The handwriting kinematic features extracted at S220 may be further analyzed to produce measures of movement fluidity and fine motor skill which may be used to compare groups of people with different health conditions and as supporting information for disease state classification.

FIG. 2B illustrates another method for analyzing handwriting kinematics, in accordance with a representative embodiment.

FIG. 2B illustrates the underlying mechanism of the recurrent region of interest feature matching algorithm by breaking down features of S220 in FIG. 2A. At S211, the method of FIG. 2B includes capturing RGB video of the subject performing handwriting. The RGB video may be captured by the electronic device 110, and either retained by the electronic device 110 or transmitted to and received by the mobile computer 130.

At S212, the method of FIG. 2B includes thresholding. As an example, to determine the location of the paper template, the OpenCV adaptive thresholding function may be used to detect lighter regions of an image frame in the RGB video. The thresholding at S212 may be performed to detect lighter regions of frames corresponding to a template of paper on which the handwriting is performed. OpenCV is a library of programming functions primarily aimed at real-time computer vision. The library is cross-platform and available for use for free under the open-source Apache 2 License.

At S213, contour detection is performed. As an example, OpenCV contour detection with default parameters may be applied to thresholded frames, and the largest contour detected may be chosen as that of the paper template.

At S214, key point selection is performed. As an example, with the detected contour from S213, the OpenCV polygonal approximation method with an epsilon value of 1% of contour arc length may be used to identify and select the 4 corners of the paper as key points.

At S215, perspective transformation is performed. From the vantage point of the camera of the electronic device 110, the polygon of the paper template may appear trapezoidal or irregular instead of rectangular. To correct for differences in camera perspective, OpenCV may be used to calculate a perspective transform matrix, which may then be used to transform the image into a top-down view of the rectangular paper. Perspective transformation may be performed on the RGB video data at S215 to obtain transformed RGB video data. The perspective transformation at S215 may include transforming frames from a RGB video corresponding to the RGB video data into top-down views using the perspective transform matrix.

At S216, the method of FIG. 2B includes obtaining pen templates and features. A template image of the pen may be captured to be used for later feature matching in coordinate extraction.

The processing from S211 to S216 may be considered pre-processing of image frames from the RGB video in a computer vision-based process. The camera of the electronic device 110 captures the RGB video, and either the electronic device 110 (FIG. 1A or FIG. 1B) or the mobile computer 130 (FIG. 1C) may be configured to collect more data than just pen position, including acquiring information about pen grip, arm pose, and/or compensatory movements with potential to diagnostic accuracy. The data from the pre-processing from S211 to S216 may be used to augment accurate pressure data from systems with the digitizing tablet 120 (FIG. 1B and FIG. 1C) or other tablet-based systems. The computer vision-based system described herein may alternatively replace use of systems with the digitizing tablet 120 or other tablet-based systems, as in FIG. 1A.

At S221, the method of FIG. 2B includes identifying features of the subject performing handwriting from captured RGB video by extracting kinematic information in the computer vision-based process. Measured tasks may include Archimedean spiral drawing (124 videos), tracing of 1's and e's (60 videos), and tracing of words (30 videos) on the PaHaW study writing template. Extracting accurate kinematic information requires pen tip x and y coordinates tagged with timestamps as the measured tasks are performed.

At S232, identified features with the greatest significance are selected in the computer vision-based process. The identified features with the greatest significance may be determined in the training of machine learning models which were trained before the method of FIG. 2B is performed. The identified features with the greatest significance are selected during the computer vision-based process from calculated kinematic features and derived features of the handwriting.

At S240, a trained machine learning model is applied to the selected subsets of identified features. The trained machine learning model uses classifiers trained to input the identified features with the greatest significance, and to output a health determination in the computer vision-based process.

At S250, a health state of the subject is determined in the computer vision-based process. For example, the health state of the subject may be qualitatively determined or quantitatively determined at S250. The health state may, for example, be a neurogenerative health state. The health state of the subject is output by the machine learning applied at S240.

As set forth above, analyzing in FIG. 2B may include analyzing transformed RGB video data for handwriting kinematic characteristics based on features extracted from the RGB video data. The analyzing results in determining a health state including newly diagnosing a disease or updating progression of a previously-diagnosed disease.

A detailed explanation of the computer vision-based process from S211 to S250 is provided with respect to FIG. 3 below, and an explanation of the training of an example machine learning model is provided with respect to FIG. 4A, FIG. 4B, FIG. 4C and FIG. 4D below.

FIG. 2C illustrates another method for analyzing handwriting kinematics, in accordance with a representative embodiment.

The method of FIG. 2C may be considered an overview of FIG. 3 as described below. In FIG. 2C, the method starts with capturing and preprocessing data at S291.

At S292, one or more frame(s) are analyzed to extract data in a computer vision-based process.

At S293, the method of FIG. 2C includes outputting and classifying results of the analysis in the computer vision-based process.

FIG. 3 illustrates a process for analyzing handwriting kinematics, in accordance with a representative embodiment.

In FIG. 3, the computer vision-based process implemented by a computer vision-based system for data extraction from videos consists of three stages: (1) preprocessing, (2) data extraction, and (3) outputs for classification. Particular emphasis is placed on the preprocessing in stage 1 and the coordinate extraction with recurrent region of interest feature matching algorithm in stage 2 at B4A, B4B, B4C, B4D, B4E and B3.

In FIG. 3, the preprocessing at stage 1 includes using a phone camera at A1 to capture and produce video frames at A2. The video frames are processed at A3 with processing including thresholding, contour detection, key point selection and/or perspective transformation. At A4, the preprocessing includes identifying paper-focused video frames. At A4, the preprocessing includes obtaining a pen template and features. In the preprocessing at stage 1 shown in FIG. 3, video frames at A2 are prepared for data extraction using thresholding, contour detection, and key point selection, followed by a perspective transform at A3 and capture of a pen template image at A5.

The data extraction at stage 2 includes detecting hand landmarks from the video frames at B1A and inputting the detected hand landmarks to a trained support vector machine at B1B. Data from the video frames at A2 is also input to a trained convolutional neural network (CNN) at B2A, and the output of the convolutional neural network is applied to a Gaussian kernel filter at B2B. The paper-focused video frames from A4 are analyzed for likely two-dimensional locations of a tip of a pen at B3. The likely two-dimensional locations of a tip of a pen and the pen template and features from A5 are subject to a process for weighted feature matching at B4A, and the output of the weighted features matching at B4A is input to a process for identifying a pen tip region of interest at B4B. The identified pen tip region of interest from B4B is subject to a detail enhancement process at B4C, to result in detection of the precise pen tip at B4D. The X coordinates and Y coordinates of the pen tip are determined at B4E, and input with the paper-focused video frames from the preprocessing at A4 to the analysis of the likely pen location at B3.

In the data extraction at stage 2 in FIG. 3, a recurrent process (i.e., a loop) is performed from the likely pen location at B3 to the analysis of the likely pen location at B4E. In the recurrent process, the paper-focused video frames from the preprocessing at A4 are also input to the analysis for the likely pen location at B3. Additionally, the pen template and features from the preprocessing at A5 are input to the weighted feature mapping at B4A. The coordinate extraction phase of stage 2 in FIG. 3 consists of tracking of the pen-tip from B4B to B4E using perspective-transformed images of the paper template from A4, using a recurrent approach to produce a region of interest for pen tip location at B4A and B4B. Accordingly, analyzing may include recurrently extracting coordinates of a tip of a pen used in the handwriting using perspective-transformed frames of the paper template and a template image of the pen, to produce regions of interest for a location of a tip of the pen.

Feature matching is used to determine a region of interest for the pen in each frame based on the original captured pen template image. The region of interest is then sharpened using OpenCV's detail enhance method, and blurred using a median filter with a size of 11. OpenCV's threshold is then applied to increase contrast between the pen tip and the background, followed by contour detection to outline the pen tip geometry in the image and enable precise detection of the pen tip.

With these extracted coordinate data and the known, consistent capture rate of cameras, kinematic features such as speed, acceleration, and jerk can be calculated. As the next frame is processed, the previous position of the pen and calculated kinematic information can be used to decrease the search area for the pen tip with feature matching, implementing a recurrent region of interest feature matching algorithm. This modification makes this tracking algorithm less computationally expensive and also more accurate, as it has a smaller search area and is less prone to single-frame errors caused by vision jitter and varying lighting conditions.

In stage 3 in FIG. 3, the pen tip is classified at C1 based on the output of the trained support vector machine from B1B. The Gaussian kernel filter at B2B outputs pen tip pressure data at C2 based on the output from the Gaussian kernel filter at B2B. Position, speed, velocity, acceleration and jerk are determined at C3 based on the X coordinates and Y coordinates of the pen tip from B4E, and the output is used to derive features including standard deviation, mean, number of relative extrema etc. at C4. The derivation of features at C4 is also based on the precise pen tip identification at B4D.

The outputs from C1, C2, C3 and C4 in stage 3 are all input to a trained ensemble classifier at C5. The output of the ensemble classifier may be a diagnostic determination such as an Alzheimer's Disease diagnosis, a Parkinson's disease diagnosis, a mild cognitive impairment diagnosis, or a healthy determination. The ensemble classifier may include a neural network, a support vector machine, and random forest. Each of the neural network, the support vector machine and the random forest may is configured to prediction votes for the subject, and an outcome with the most prediction votes may be chosen.

Results from the framework shown in FIG. 3 may be compared to results of a digitizing tablet. To assess accuracy of kinematic data produced by the computer vision-based system described herein, the timestamps associated with the data from the computer vision-based system may be matched in a pairwise fashion to digitizing tablet data with the closest timestamp. The aligned time series data may then be used to calculate errors and determine accuracy of the computer vision-based system.

Mean absolute error (MAE) for position may be calculated using the following formula across the entire length i of each time series, where (xᵢ, yᵢ) represent digitizing tablet coordinate data, and (xᵢ′, yᵢ′) represent vision-based data:

$$\\ M A E = {\sum }_{n = 1}^{i} \sqrt{{( {x}_{i} - {x}_{i}^{′} )}^{2} + {( {y}_{i} - {y}_{i}^{′} )}^{2}}$$

Kinematic features of speed, acceleration, and jerk may be calculated using symmetrical differences using the following formulas:

$$\\ {s}_{i} = \frac{\sqrt{{( {x}_{i + 1} - {x}_{i - 1} )}^{2} + {( {y}_{i + 1} - {y}_{i - 1} )}^{2}}}{{t}_{i + 1} - {t}_{i - 1}}$$

$$\\ {a}_{i} = \frac{{s}_{i + 1} - {s}_{i - 1}}{{t}_{i + 1} - {t}_{i - 1}}$$

$$\\ {j}_{i} = \frac{{a}_{i + 1} - {a}_{i - 1}}{{t}_{i + 1} - {t}_{i - 1}}$$

The PaHaW dataset may be used to demonstrate the practicality of computer vision-based data in discriminative neurodegenerative disease classification. The collected coordinate information in the dataset may be down-sampled from the 100 Hz collected by digitizing tablets to 30 Hz and 60 Hz, insofar as 30 Hz and 60 Hz are typical frame rates produced by cameras. The adjusted data may then be used to calculate kinematic features, including speed, acceleration, and jerk. For example, a total of 176 derived features may be produced, including mean, minimum, maximum, standard deviation, and number of extrema for profiles of each kinematic feature during a handwriting task. These features may then be tested for statistical significance using t-tests to produce the final feature set, consisting of the features with p-values less than 0.10 for each data capture rate.

Quantitative comparisons of the computer vision-based system for quantifying fine motor kinematic data from videos to the digitizing tablet are summarized in Tables I and II. Most important to note are the position MAEs, which are less than 0.5 mm for both spirals (n=124) and writing (n=90). Furthermore, the speed and acceleration MAEs were under 1.1% for spiral tasks (n=124), and under 2% for handwriting tasks (n=90). FIG. 4 shows a graphical comparison of these kinematic features for representative Archimedean spiral and handwriting tasks, demonstrating the nearly identical kinematic information captured by the computer vision-based approach described herein compared to the digitizing tablet.

TABLE I
ACCURACY OF VISION-BASED
KINEMATIC DATA - SPIRAL

| Data | MAE | % MAE | 95% CI |
|---|---|---|---|
| Position | 0.48 mm | N/A | ±0.088 mm |
| x-coordinate | 0.29 mm | N/A | ±0.088 mm |
| y-coordinate | 0.31 mm | N/A | ±0.041 mm |
| Speed | 1.54 mm/s | 1.05% | ±0.169 mm/s |
| Acceleration | 0.93 mm/s² | 1.08% | ±0.177 mm/s² |
| Jerk | 4.16 mm/s³ | 2.76% | ±0.837 mm/s³ |

TABLE II
ACCURACY OF VISION-BASED
KINEMATIC DATA - WRITING

| Data | MAE | % MAE | 95% CI |
|---|---|---|---|
| Position | 0.40 mm | N/A | ±0.055 mm |
| x-coordinate | 0.24 mm | N/A | ±0.056 mm |
| y-coordinate | 0.27 mm | N/A | ±0.041 mm |
| Speed | 2.39 mm/s | 1.95% | ±0.286 mm/s |
| Acceleration | 1.88 mm/s² | 1.78% | ±0.399 mm/s² |
| Jerk | 3.71 mm/s³ | 1.87% | ±0.924 mm/s³ |

FIG. 4A illustrates an overview of a machine learning system for analyzing handwriting kinematics, in accordance with a representative embodiment.

The machine learning system of FIG. 4A consists of (1) an inputs and preprocessing stage, a (2) ensemble learning and classification stage, and a (3) diagnostic assessment stage. The (1) inputs and preprocessing stage is to calculate kinematic and derived features and choose those with the greatest significance. The (2) ensemble learning and classification stage is an ensemble classifier for training and testing. The (3) diagnostic assessments stage is to output a diagnostic assessment.

FIG. 4B illustrates a first stage of a machine learning system for analyzing handwriting kinematics, in accordance with a representative embodiment.

In FIG. 4B, pen tip x, y coordinates and associated timestamps are input to Gaussian and median filters. The output of the Gaussian and median filters include speed, acceleration and jerk. Metrics calculated for speed, acceleration and jerk may include standard deviation, number of extrema, mean, maximum and minimum. The first stage performs T-tests for significance, and outputs the results to the second stage.

FIG. 4C illustrates a second stage of a machine learning system for analyzing handwriting kinematics, in accordance with a representative embodiment.

In FIG. 4C, an ensemble classifier, consisting of a neural network, support vector machine, and random forest may be trained on the most significant data output from the first stage using 10-fold cross validation to prevent overfitting. Each machine learning structure casts a prediction vote for the patient, and the outcome with the most votes (Parkinson's Disease or healthy control) is chosen in the classification voting.

FIG. 4D illustrates a third stage of a machine learning system for analyzing handwriting kinematics, in accordance with a representative embodiment.

In FIG. 4D, outputs of the classification voting in FIG. 4C include a healthy control or a diagnosis of Parkinson's disease. However, as should be clear from the teachings herein, the potential classifications of an ensemble classifier are not limited to two results, and are not limited to particular illnesses such as Parkinson's disease. Rather, trained artificial intelligence will provide outputs reflective of training.

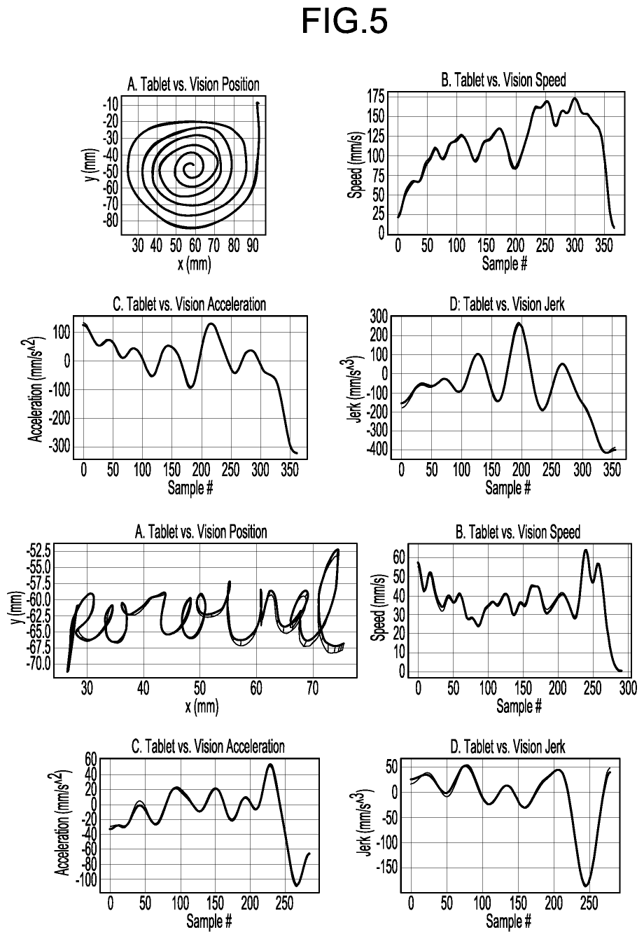

FIG. 5 illustrates a relative comparison of a computer vision-based system for analyzing handwriting kinematics to a digitizing tablet control system for analyzing handwriting kinematics, in accordance with a representative embodiment.

In FIG. 5, results of a computer vision-based data collection system are compared relative to results of digitizing tablet control. The results are demonstrated by overlaid graphs of extracted kinematic features in an Archimedean spiral and word writing task: (A) Pen tip position (B) Pen tip speed (C) Pen tip acceleration (D) Pen tip jerk. Graphs (B), (C) and (D) display narrow 95% confidence intervals. Note the slight position drift in the word writing task, which is due to the pen's increasing distance from the camera.

Accuracy of the ensemble learning classification system described herein may be assessed using data down-sampled to three rates of capture: the tablet-collected 100 Hz, and down-sampled values of 60 and 30 Hz to simulate vision-based data. The findings are shown in Table III below.

An accuracy of 74% (n=75) may be achieved with the 60 Hz capable of capture by many modern, accessible computer vision-based systems, which is nearly identical to the 75% (n=75) achievable with 100 Hz offered by digitizing tablet data and very similar sensitivity and specificity values. Furthermore, even at a capture rate of 30 Hz, which is attainable with nearly all commodity cameras, an accuracy of 71% (n=75) may be achieved in distinguishing Parkinson's Disease patients from healthy controls, with slightly lower sensitivity at specificity values compared to the higher frequencies. However, an improved sensitivity may provide for improved screening. An accuracy of 79-80% may be achieved with a computer vision-based systems with down-sampled capture rates higher than 60 Hz.

TABLE III
DIAGNOSTIC ASSESSMENT PERFORMANCE
BY DATA CAPTURE RATES

| Frequency (Hz) | Accuracy | Sensitivity | Specificity |
|---|---|---|---|
| 30 | 71% | 75% | 65% |
| 60 | 74% | 79% | 70% |
| 100 | 75% | 80% | 72% |

The results of the study reflected in Table III demonstrate the practicality of a framework using commodity cameras, such as those in smartphones, to accurately quantify kinematic information of fine motor movements with computer vision algorithms. The significance of this is further compounded by the accuracy achieved in classifying Parkinson's Disease patients and healthy controls using data at frequencies that can be captured by commodity cameras, with accuracy rivaling that of the current clinical diagnostic process.

The computer vision-based aspects of the systems described herein, in combination with modern widespread access to cameras with capability of capturing these data in mobile phones and other devices, may enable wider access to neurodegenerative disease diagnostic screening, especially in lower-income populations and resource-poor health systems. Furthermore, the system's at-home accessibility enhances long-term monitoring of disease state, including treatment effects, clinical deterioration, and disease progression, via telemedicine. This ease of use also allows for larger-scale data collection of handwriting movements of patients with neurodegenerative diseases as well as healthy controls to develop and improve understanding of differences between these groups and increase diagnostic accuracy.

The descriptions herein have focused primarily on uses for neurodegenerative disease diagnostic assessment. However, the framework for computer vision-based kinematic analysis of fine motor movement may be utilized to screen for any health conditions in which biomarkers are displayed in handwriting movements, including strokes, early developmental disorders (e.g., dysgraphia), and arthritis. An accessible and easy-to-use tool for assessing these movements is a necessary step to better understand these biomarkers' significance in the diagnostic process, while the resultant expedited diagnostic processes have potential to improve treatment outcomes for these conditions.

As set forth herein, an accessible, vision-based system is capable of analyzing fine motor movements in handwriting tasks to provide neurodegenerative diseases diagnostic assessments. The experimental results show that accurate quantification of fine motor movement kinematic features is possible with low-cost commodity cameras. The inventive concepts described herein demonstrate that kinematic data sampled at frequencies commonly found in commodity cameras is viable for distinguishing between neurodegenerative disease patients and healthy controls on the PaHaW data set, with high sensitivity and specificity achieved in diagnostic assessments. This system can be used to increase neurodegenerative diseases diagnostic access in lower-income populations and resource-poor health systems, provide a long-term disease monitoring solution through telemedicine, and offer a quantifiable tool to support clinical diagnosis of neurodegenerative diseases.

The accuracy of the computer vision-based systems and methods described herein may be tested with additional data collected for quantifying kinematic information. Additional data collection may also allow for testing of the significance of vision-specific features such as pen grip and body pose during writing, and exploring the estimation of pen pressure from video data.

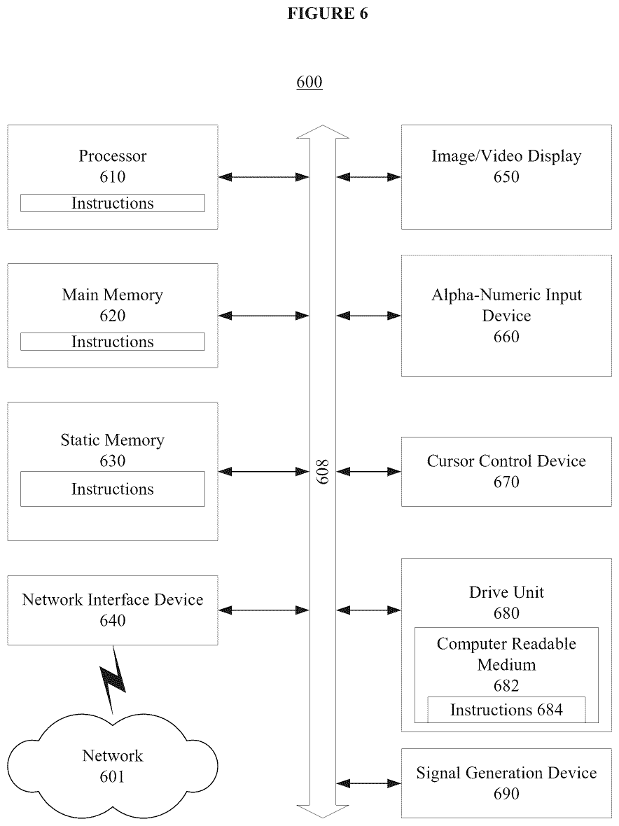

FIG. 6 illustrates a computer system, on which a method for analyzing handwriting kinematics is implemented, in accordance with another representative embodiment.

Referring to FIG. 6, the computer system 600 includes a set of software instructions that can be executed to cause the computer system 600 to perform any of the methods or computer-based functions disclosed herein. The computer system 600 may operate as a standalone device or may be connected, for example, using a network 601, to other computer systems or peripheral devices. In embodiments, a computer system 600 performs logical processing based on digital signals received via an analog-to-digital converter.

In a networked deployment, the computer system 600 operates in the capacity of a server or as a client user computer in a server-client user network environment, or as a peer computer system in a peer-to-peer (or distributed) network environment. The computer system 600 can also be implemented as or incorporated into various devices, such as the devices described herein including a stationary device or a mobile device, a mobile computer, a laptop computer, a tablet computer, or any other machine capable of executing a set of software instructions (sequential or otherwise) that specify actions to be taken by that machine. The computer system 600 can be incorporated as or in a device that in turn is in an integrated system that includes additional devices. In an embodiment, the computer system 600 can be implemented using electronic devices that provide voice, video or data communication. Further, while the computer system 600 is illustrated in the singular, the term “system” shall also be taken to include any collection of systems or sub-systems that individually or jointly execute a set, or multiple sets, of software instructions to perform one or more computer functions.

As illustrated in FIG. 6, the computer system 600 includes a processor 610. The processor 610 executes instructions to implement some or all aspects of methods and processes described herein. The processor 610 is tangible and non-transitory. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The processor 610 is an article of manufacture and/or a machine component. The processor 610 is configured to execute software instructions to perform functions as described in the various embodiments herein. The processor 610 may be a general-purpose processor or may be part of an application specific integrated circuit (ASIC). The processor 610 may also be a microprocessor, a microcomputer, a processor chip, a controller, a microcontroller, a digital signal processor (DSP), a state machine, or a programmable logic device. The processor 610 may also be a logical circuit, including a programmable gate array (PGA), such as a field programmable gate array (FPGA), or another type of circuit that includes discrete gate and/or transistor logic. The processor 610 may be a central processing unit (CPU), a graphics processing unit (GPU), or both. Additionally, any processor described herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems. The term computing device should also be interpreted to include a collection or network of computing devices each including a processor or processors. Programs have software instructions performed by one or multiple processors that may be within the same computing device or which may be distributed across multiple computing devices.

The computer system 600 further includes a main memory 620 and a static memory 630, where memories in the computer system 600 communicate with each other and the processor 610 via a bus 608. Either or both of the main memory 620 and the static memory 630 may store instructions used to implement some or all aspects of methods and processes described herein. Memories described herein are tangible storage mediums for storing data and executable software instructions and are non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The main memory 620 and the static memory 630 are articles of manufacture and/or machine components. The main memory 620 and the static memory 630 are computer-readable mediums from which data and executable software instructions can be read by a computer (e.g., the processor 610). Each of the main memory 620 and the static memory 630 may be implemented as one or more of random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, blu-ray disk, or any other form of storage medium known in the art. The memories may be volatile or non-volatile, secure and/or encrypted, unsecure and/or unencrypted.

“Memory” is an example of a computer-readable storage medium. Computer memory is any memory which is directly accessible to a processor. Examples of computer memory include, but are not limited to RAM memory, registers, and register files. References to “computer memory” or “memory” should be interpreted as possibly being multiple memories. The memory may for instance be multiple memories within the same computer system. The memory may also be multiple memories distributed amongst multiple computer systems or computing devices.

As shown, the computer system 600 further includes a video display unit 650, such as a liquid crystal display (LCD), an organic light emitting diode (OLED), a flat panel display, a solid-state display, or a cathode ray tube (CRT), for example. Additionally, the computer system 600 includes an input device 660, such as a keyboard/virtual keyboard or touch-sensitive input screen or speech input with speech recognition, and a cursor control device 670, such as a mouse or touch-sensitive input screen or pad. The computer system 600 also optionally includes a disk drive unit 680, a signal generation device 690, such as a speaker or remote control, and/or a network interface device 640.

In an embodiment, as depicted in FIG. 6, the disk drive unit 680 includes a computer-readable medium 682 in which one or more sets of software instructions 684 (software) are embedded. The sets of software instructions 684 are read from the computer-readable medium 682 to be executed by the processor 610. Further, the software instructions 684, when executed by the processor 610, perform one or more steps of the methods and processes as described herein. In an embodiment, the software instructions 684 reside all or in part within the main memory 620, the static memory 630 and/or the processor 610 during execution by the computer system 600. Further, the computer-readable medium 682 may include software instructions 684 or receive and execute software instructions 684 responsive to a propagated signal, so that a device connected to a network 601 communicates voice, video or data over the network 601. The software instructions 684 may be transmitted or received over the network 601 via the network interface device 640.

In an embodiment, dedicated hardware implementations, such as application-specific integrated circuits (ASICs), field programmable gate arrays (FPGAs), programmable logic arrays and other hardware components, are constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

As set forth above, a computer vision-based system and method may be configured to capture handwriting kinematic information. A method for diagnosing neurodegenerative diseases and a diagnostic tool for diagnosing neurodegenerative diseases may be implemented with the computer vision-based systems and methods described herein. A diagnostic tool and method for screening health conditions may be provided in which biomarkers are displayed in handwriting movements captured and processed with the computer vision-based systems and methods described herein. A non-transient computer-readable medium may store software which, when executed by a processor, causes the processor to capture and process the handwriting kinematic information.

Although computer vision for analyzing handwriting kinematics has been described with reference to several exemplary embodiments, it is understood that the words that have been used are words of description and illustration, rather than words of limitation. Changes may be made within the purview of the appended claims, as presently stated and as amended, without departing from the scope and spirit of computer vision for analyzing handwriting kinematics in its aspects. Although computer vision for analyzing handwriting kinematics has been described with reference to particular means, materials and embodiments computer vision for analyzing handwriting kinematics is not intended to be limited to the particulars disclosed; rather computer vision for analyzing handwriting kinematics extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed

I claim:

1. A system for analyzing handwriting kinematics, comprising:
a memory that stores executable instructions;
a processor that executes the executable instructions,
wherein, when executed by the processor, the executable instructions cause the system to implement a process that includes:
receiving RGB video data of a subject performing handwriting;
extracting features from the RGB video data, and
analyzing the RGB video data for handwriting characteristics based on the features extracted from the RGB video data.

2. The system of claim 1,
wherein the handwriting characteristics comprise characteristics of handwriting kinematics, and
wherein a process implemented when the instructions are executed by the processor further comprises:
performing perspective transformation on the RGB video data to obtain transformed RGB video data,
wherein the features extracted from the RGB video data are extracted from the transformed RGB video data, and
wherein the analyzing comprises analyzing the transformed RGB video data for the characteristics of handwriting kinematics based on the features extracted from the RGB video data.

3. The system of claim 2,
wherein the RGB video data include video of a pen used to perform the handwriting, and
wherein the perspective transformation comprises transforming frames from a RGB video corresponding to the RGB video data into top-down views using a perspective transform matrix.

4. The system of claim 3, wherein the analyzing comprises preprocessing the RGB video data using thresholding, contour detection, and key point selection to capture images of the pen used to perform the handwriting.

5. The system of claim 4,
wherein the thresholding includes detecting lighter regions of frames corresponding to a template of paper on which the handwriting is performed, and
wherein the contour detection is applied to thresholded frames to select a contour as a paper template, and then identify corners of the paper template.

6. The system of claim 1,
wherein a process implemented when the instructions are executed by the processor further comprises:
applying the features extracted from the RGB video data to processing by an ensemble classifier to classify the handwriting performed by the subject, and
wherein the ensemble classifier comprises a plurality of machine learning models which each apply machine learning to the features extracted from the RGB video data.

7. The system of claim 6, wherein the analyzing comprises calculating kinematic features and derived features of the handwriting and selecting subsets of the kinematic features and the derived features determined to have greatest significance in training of the machine learning models.

8. The system of claim 1, further comprising:
a commodity camera which captures the RGB video data,
wherein the memory, the processor and the commodity camera are integrated into a mobile device.

9. The system of claim 1, wherein a process implemented when the instructions are executed by the processor further comprises:
quantitatively determining a health state of the subject performing handwriting by newly diagnosing presence of a disease, wherein the health state comprises a neurogenerative health state.

10. The system of claim 1, wherein a process implemented when the instructions are executed by the processor further comprises:
quantitatively determining a health state of the subject performing handwriting by determining change over time of a previously-diagnosed disease, wherein the health state comprises a neurogenerative health state.

11. The system of claim 1, wherein a process implemented when the instructions are executed by the processor further comprises:
identifying, from the RGB video data, characteristics of the subject as the subject is performing the handwriting.

12. The system of claim 1, wherein a process implemented when the instructions are executed by the processor further comprises:
receiving, from a digitizing tablet, data of characteristics of the handwriting performed by the subject, and
augmenting the analyzing with the data of characteristics of the handwriting performed by the subject.

13. The system of claim 1,
wherein the RGB video data is derived from an RGB video captured at a frame rate below 61 Hz.

14. The system of claim 1,
wherein the analyzing comprises extracting kinematic information including two-dimensional locations of a tip of a pen used in the handwriting and timestamps for each reading of the two-dimensional locations.

15. The system of claim 1, wherein the analyzing comprises recurrently extracting coordinates of a tip of a pen used in the handwriting using perspective-transformed frames of the paper template and a template image of the pen, to produce regions of interest for a location of a tip of the pen.

16. The system of claim 1, wherein a process implemented when the instructions are executed by the processor further comprises:
feature matching to determine a region of interest for a pen used in performing the handwriting, and sharpening the region of interest for the pen in each frame to precisely detect a tip of the pen.

17. The system of claim 6,
wherein the ensemble classifier consists of a neural network, support vector machine, and random forest, and each of the neural network, the support vector machine and the random forest is configured to cast prediction votes for the subject, and an outcome of the prediction votes with most votes is chosen.

18. A method for analyzing handwriting kinematics, comprising:
receiving RGB video data of a subject performing handwriting;
extracting features from the RGB video data, and
analyzing the RGB video data for handwriting characteristics, using a machine learning model, based on the features extracted from the RGB video data.

19. The method of claim 18, further comprising:
performing perspective transformation on the RGB video data to obtain transformed RGB video data,
wherein the perspective transformation comprises transforming frames from a RGB video corresponding to the RGB video data into top-down views using a perspective transform matrix

20. A computer-readable medium that stores instructions which, when executed by a processor, implement a process that includes:
receiving RGB video data of a subject performing handwriting;
extracting features from the RGB video data, and
analyzing the RGB video data for handwriting characteristics based on the features extracted from the RGB video data.
